(19) United States
(12) Patent Application Publication
(10) Pub. No.: US 2009/0198169 A1
(43) Pub. Date: Aug. 6, 2009

(54) DEVICE AND METHOD FOR IRRADIATING BLOOD

(75) Inventor: Todd John Baumeister, Edmonds, WA (US)

Correspondence Address: SEED INTELLECTUAL PROPERTY LAW GROUP PLLC, 701 FIFTH AVE, SUITE 5400, SEATTLE, WA, 98104, US

(73) Assignee: PRISM BIOTECH, INC., Edmonds, WA (US)

(21) Appl. No.: 12/327,685

(22) Filed: Dec. 3, 2008

Related U.S. Application Data
(63) Continuation of application No. 10/570,524, filed on Nov. 14, 2006, now abandoned.
(60) Provisional application No. 60/592,063, filed on Jul. 29, 2004.
(60) Provisional application No. 60/500,366, filed on Sep. 4, 2003.

(51) Int. Cl. A61M 1/36

(52) U.S. Cl. 604/6.08

(57) ABSTRACT

A device for irradiating blood is provided, the device including a chamber having an elongate configuration defining a hollow interior, the chamber formed of reflective material, a tubular cassette sized and shaped to be received in the interior of the chamber, the tubular cassette having an inside diameter sized and shaped to receive a reflective core having an elongate configuration with an exterior diameter smaller than the interior diameter of the cassette to provide a space for receiving and holding blood stationary, and an array of light-emitting members arranged on the chamber for emitting light of at least one wavelength into the interior of the chamber for irradiating and treating the blood inside the cassette.

### CROSS-REFERENCE TO RELATED APPLICATION

This application is a continuation of U.S. patent application Ser. No. 10/570,524, filed Nov. 14, 2006, which is a National Stage of PCT/US2004/028003, filed Aug. 27, 2004, and claims the benefit under 35 U.S.C. § 119(e) of U.S. Provisional Application No. 60/592,063, filed Jul. 29, 2004 and U.S. Provisional Application No. 60/500,366, filed Sep. 4, 2003, where these applications are incorporated herein by reference in their entireties.

### BACKGROUND OF THE INVENTION

1. Field of the Invention

This invention relates generally to a method of irradiating blood and to an irradiation chamber wherein blood is irradiated with various wavelengths of light for the purpose of altering its immunologic status, and more particularly to a chamber of this type in which a stationary fixed amount of blood is measured and displayed for even exposure to a light source of very low heat output, low intensity, and rapid activation and deactivation.

This invention shall also relate to a light source specifically designed to couple with the chamber for the purpose of irradiating the blood contained within it, and which has a light source.

2. Description of the Related Art

The irradiation of blood as a means to alter its immunologic status has been researched since its inception by Knott in 1928. This has always included the extraction of blood and passing it continuously through a chamber while it is irradiated, usually with ultraviolet light. This light is of low intensity in the Knott device. In other methods activating chemicals are used, and higher intensity light is used with devices for clearing the blood of pathogens for blood banking. Chambers for this purpose need to have baffles or be constructed of tubing such that the blood can churn within the chamber so that the greatest amount of blood is exposed to the UV light. This construction was required because of the lack of availability of an ultraviolet light source that was of low heat output and that could be rapidly turned on and off.

Of prior art interest in regard to such treatment is the blood irradiation chamber disclosed in an article by E.K. Knott in the August 1948 issue (Vol. LXXVI-No. 5) of the American Journal of Surgery, entitled “Development of Ultraviolet Blood Irradiation.” In the disclosed device, extracorporal blood is pumped through a quartz chamber two inches in diameter and one inch in thickness. This chamber contains baffles so that the blood is churned to expose as many elements to the mercury-vapor lamp source as possible. Patents that show Knott-type blood chambers include U.S. Pat. Nos. 1,683,877; 2,309,124; 2,308,516; 2,314,281; and 6,312,593.

The failing of the Knott-type devices is that they have light sources that are hot, noisy, and require warm up before use. This makes placebo treatments difficult to accomplish, thus limiting research. This also leads to inaccuracy in calculating dosing for research purposes.

Another failing of the prior devices is that blood is moved through an exposure chamber during exposure to the light source. Moving volumes lead to inaccuracy when dosages are calculated.

Another failing of the prior devices is that they utilize baffles to churn the blood within their chamber, resulting in uncertainty as to whether all elements in the blood have been properly exposed. Unequal exposure leads to inaccuracy when dosing is calculated.

### BRIEF SUMMARY OF THE INVENTION

In view of the foregoing, the disclosed embodiments of the present invention provide for a chamber whereby light can equally irradiate a stationary quantified amount of blood extracorporally, and a light source coupled to the chamber whereby the chamber can be safely irradiated with a light generator that can be quickly activated and deactivated while remaining cool and quiet enough to permit placebo treatment.

In accordance with another embodiment of the invention, a chamber is provided that is configured to be easily sterilized and reused by the same patient/subject.

In accordance with another embodiment of the invention, control over the light source is provided whereby its duration, intensity, and wavelength can be easily and quickly adjusted.

In accordance with one embodiment of the invention, a device for subjecting a stationary quantity of blood to light for the purpose of altering the immune function of the patient is provided. The device includes a chamber formed by a window of a quartz plate and a back formed of hard plastic having an inlet port and an outlet port that communicate with the chamber, including a stopcock valve adjacent the outlet port to retain blood in the chamber and to selectively permit the entry of fluids into the chamber when treated blood exits the chamber back to the patient via the inlet port; and a housing for directing light from a light source to the chamber, the housing including a holder made of plastic having a slot to receive the chamber, a clamp to hold the chamber in the housing, and a mounting with a light source board at an end opposite the holder, and a reflective inner surface to reflect light from the light source to the chamber.

In accordance with another aspect of the foregoing embodiment of the invention, the light source board includes a printed circuit board having an array of light emitting diodes. Preferably at least one of the diodes in an ultraviolet light emitting diode.

In accordance with another aspect of the foregoing embodiment of the invention, a microprocessor or computer system is provided that is coupled to the light source to control the lighting of the diodes such that the wavelength of emitted light can be varied or combined to treat various pathological conditions in the blood.

In accordance with another aspect of the invention, a method of treating a measured and stationary amount of blood from a patient is provided. The method includes receiving blood intravenously from a patient at an inlet port of a chamber by force of the intravenous blood pressure; filling the chamber with the patient's blood from the inlet port at the bottom of the chamber to a valve at an outlet port at the top of the chamber; exposing the blood in the chamber to a light source for the purpose of altering the immune function of the blood of the patient; opening the valve at the outlet port to introduce fluids into the chamber through the outlet port with sufficient force to return the blood back to the patient intravenously and flushing the chamber with the fluid; and repeating the foregoing steps as desired.

In accordance with another embodiment of the invention, a device for irradiating blood is provided that includes an elongate reflective chamber, preferably of a circular cross-sectional configuration, although it may have other configurations, such as octagonal, hexagonal, pentagonal, or the like. The chamber has a hollow interior to which access is provided by an access panel hingedly attached as part of the chamber wall. An elongate tube sized and shaped to be received in the interior of the chamber is provided, the tube having an inside diameter and a reflective core, preferably hollow, placed therein having an exterior diameter that is smaller than the interior diameter of the tube to provide a space for holding the blood stationary; and an array of light-emitting members mounted on the chamber for providing irradiating light of one or more wavelengths to the interior of the chamber for irradiating the blood. Ideally, the tube and the hollow reflective core also have circular cross-sectional configurations to provide maximum reflectivity.

As will be readily appreciated from the foregoing, the tube with hollow core, referred to as a cassette, is disposable to provide safety to healthcare providers. It is also detachable from the blood withdrawing apparatus attached to the patient so as to reduce the risk or eliminate the risk of electrocution to the patient. In addition, the designs of the present invention maintain the blood in a fixed or stationary condition during irradiation. LEDs provide instant cooler light and the ability to select wavelengths. A computer program provides control to the LEDs, allowing double-blind studies and the transmission of data back to a computer. Energy usage is low enough to enable portability for disaster relief and field hospitals. The device is safer because light cannot escape from the unit and damage the eyes.

### BRIEF DESCRIPTION OF THE SEVERAL VIEWS OF THE DRAWINGS

For a better understanding of the invention, as well as features and advantages thereof, reference is made to the accompanying drawings wherein;

FIG. 1 is an exploded side view illustration of a blood treatment system that includes an irradiation chamber coupled with its light source in accordance with the present invention;

FIG. 2 is a side view of a chamber;

FIG. 3 is a side view of a chamber holder;

FIG. 4 is a side view of a housing;

FIG. 5 is a side view of a light source board;

FIG. 6 is a front view of the chamber of the present invention;

FIG. 7 is a front view of the holder of the present invention;

FIG. 8 is a side view of another embodiment of the invention;

FIG. 9 is a cross-sectional view taken along lines 9-9 of the embodiment of FIG. 8; and

FIG. 10 is an alternative embodiment of the design of FIGS. 8 and 9.

### DETAILED DESCRIPTION OF THE INVENTION

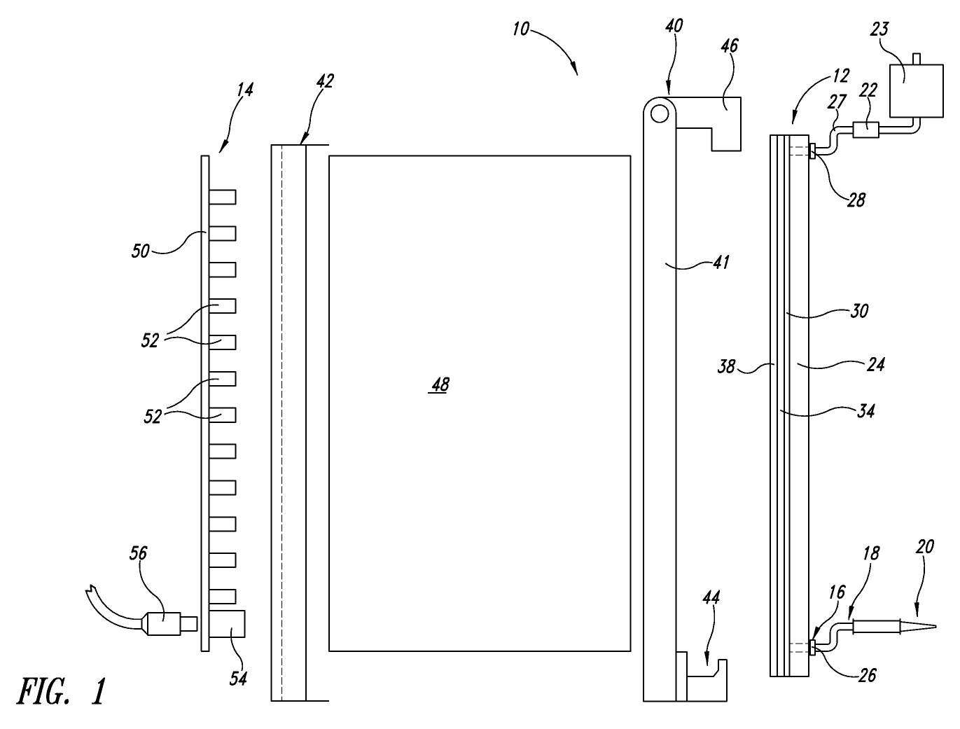

Referring first to FIG. 1, shown therein is a system 10 for treating immune problems of a patient by affecting the immune elements within a portion of the patient's blood. The system 10 includes an irradiation chamber 12 formed in accordance with the invention and a source 14 of light radiation. An inlet port 16 is formed on the chamber 12 and is coupled by a tube 18 to a hypodermic needle 20 that is inserted into the arm of a patient for withdrawing blood from the patient and returning it after treatment.

The blood of the patient enters into the chamber 12 by gravity feed and the inherent pressure of the blood stream, whereupon it is stopped with a stopcock 22 when the chamber 12 is full. The blood in the chamber 12 is exposed to light emanating from the light source 14 to alter the immune status of the fixed amount of measured blood within the chamber 12. After exposure, the blood is then returned to the patient in a reverse direction via the same pathway. The stopcock 22 is turned to allow fluid from a hung 23 bag or bottle to enter into the chamber 12, thus forcing the blood back into the patient and rinsing the chamber 12 of blood.

As illustrated in FIG. 2, the chamber 12 has a back plate 24 that is composed of hard plastic. This back plate 24 has a Luer-type male fitting 26 for connecting the tube 18 from the patient at its lower rear area, and another Luer-type female fitting 28 is located at an upper rear area of the back plate 24 for connecting the stopcock 22 and a tube 27 to the intravenous-type fluids to be delivered to the patient after the blood in the chamber 12 is exposed to the UV light.

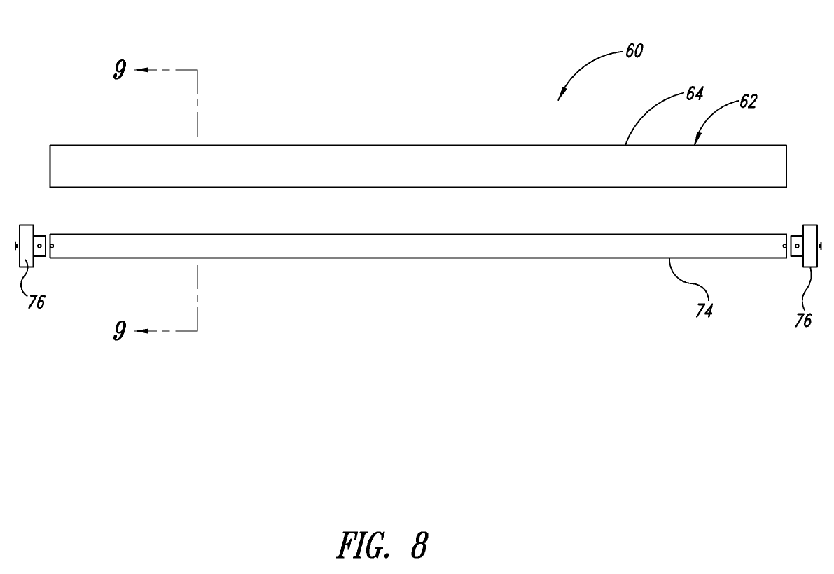

The chamber 12 has a gasket 30 formed of semisoft plastic, preferably 2 mm thick, and which has an area 32 free within it, preferably 20.0 centimeters by 25.0 centimeters. This free area 32, when a window plate 34 of quartz or other material is applied, forms a 100.0 cubic centimeter vessel 36 (shown in FIG. 6) for measuring the blood to be exposed to the UV light. The plate 34 and the gasket 30 are held to the back plate 24 with a frame 38 of hard plastic through which small screws 39 are fastened into the back plate 24 of the chamber 12.

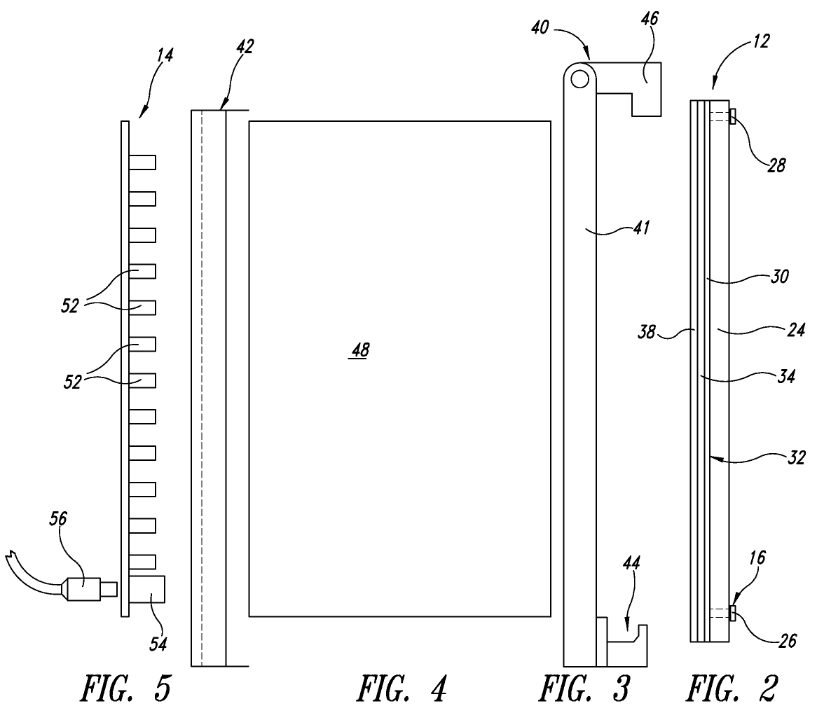

A chamber holder 40 is illustrated in FIG. 3 and is configured to hold the chamber 12 against a housing 42 for the light source 14. Preferably, the holder 40 is composed of hard plastic. The chamber 12 is placed into a slot 44 in a lower portion of the holder when a camming clamp is raised. The clamp 46 is hinged to the holder 40 (See FIG. 7), and when lowered into position over the chamber 12, by its wedge shape and its weight it holds the chamber 12 against a frame 41 of the holder 40. The holder 40, being attached to one end of the light housing 42, thus holds the chamber 42 in place for blood exposure to the light source 14 at the other end of the housing 42.

The housing 42 illustrated in FIG. 4 bears the chamber 12 and chamber holder 40 at one end and the light source 14 at the other end. The chamber 12 is composed of reflective metal sheet 48 on one surface, that surface faced into the center of the housing 42 when the metal sheet 48 is bent to form the rectangular tube-shaped housing. The length of the rectangular tube thus formed is determined by the spread of light from the light source board 14, thus aiming to maximize the exposure of the blood in the chamber 12 to the light sent from the light source 14.

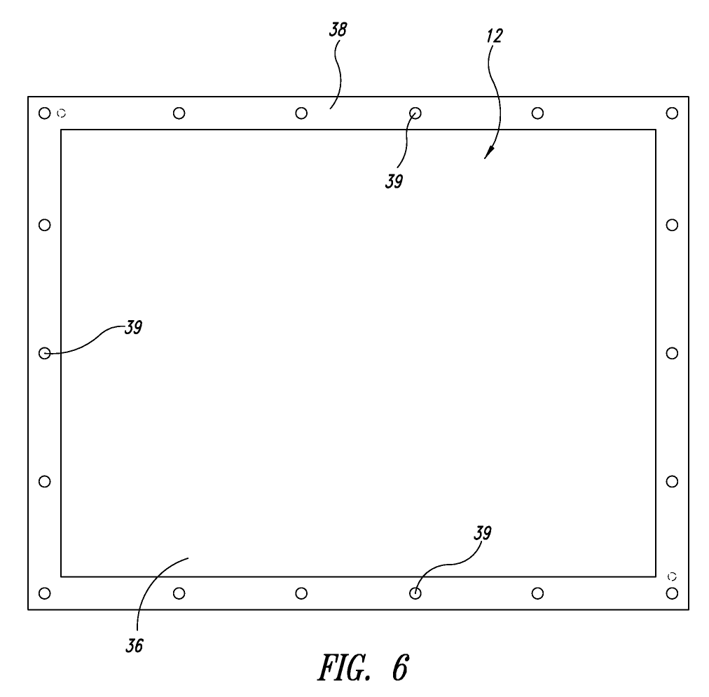

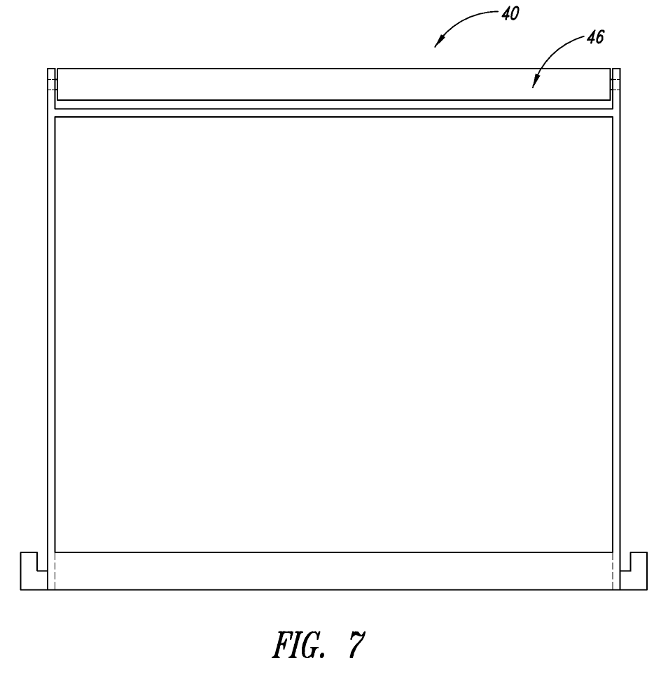

The light source 14 is mounted to a light source board 50, illustrated in FIG. 5, formed of a printed circuit type board containing an array of light emitting diodes 52. In one embodiment of the invention, light source boards 50 will have ultraviolet light emitting diodes 52. However, other arrays can contain a mixture of various wavelength light emitting diodes as the invention is tailored to treat various diseases more specifically. The board 50 has a USB connection 54 whereby it may be connected to an external computer of conventional configuration via a cable 56 for powering the light source 14 and controlling the array of light emitting diodes 52.

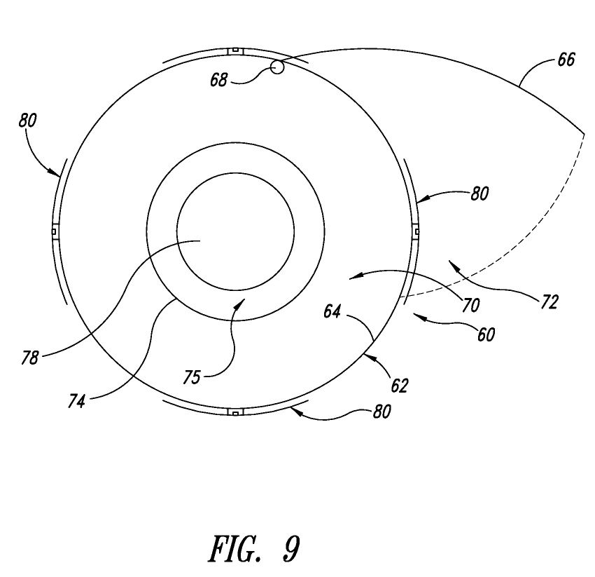

FIGS. 8 and 9 illustrate another embodiment of the invention wherein a device 60 for irradiating blood is provided. The device 60 includes a reflective chamber 62 having in this embodiment a circular cross-sectional configuration. Ideally, the chamber 62 is formed from a reflective stainless steel wall 64 having an access panel 66 mounted via a hinge 68 thereon. The access panel 66 opens to provide access to an interior 70 through a longitudinal opening 72. The device 60 further includes a quartz tube 74 having a hollow reflective core 78, preferably formed of plastic, that also has a circular cross sectional configuration sized and shaped to be received inside the reflective chamber 62. End caps 76 are placed on each end of the quartz tube to support the quartz tube inside the chamber 62 and to retain blood in a space 75 between the core 78 and the tube 74.

A plurality of LED arrays 80 are attached to the outside of the chamber 62, each having a cover (not shown). Corrugated low-voltage sheathing (not shown) will pass wires to the array. The LED arrays 80 include at least one ultraviolet LED. An opening is formed in the chamber 62 at each LED location to admit light into the chamber 62.

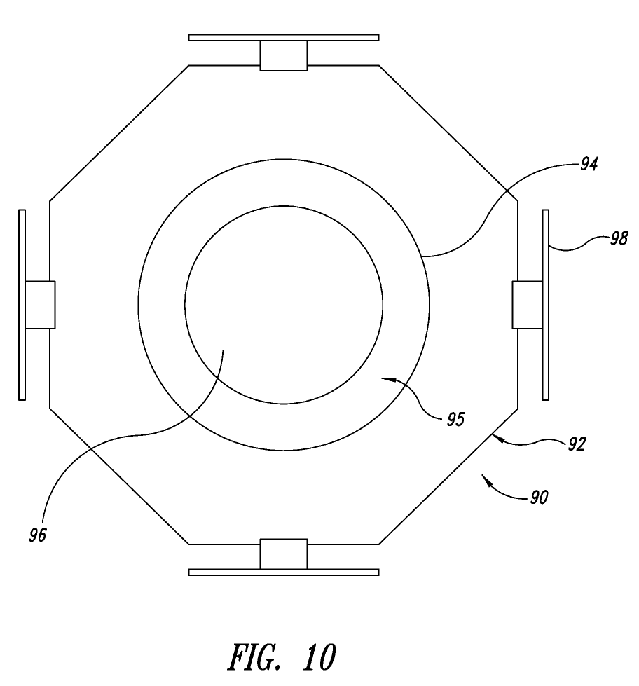

An alternative embodiment of the design shown in FIGS. 8 and 9 is illustrated in FIG. 10 in which the device 90 has a reflective chamber 72 with an octagonal cross-sectional configuration. An interior-mounted quartz tube 94 having a circular cross-sectional configuration is shown positioned inside the chamber 92. In this embodiment irradiation is provided by the LED arrays 98 arranged around the exterior of the chamber 92. Blood to be treated will be held stationary in a space 95 between the quartz tube 94 and the core 96.

This tubular design will use components that are readily commercially available, hence making them extremely inexpensive by comparison to custom-made components. In addition, this gives the device disposable characteristics, thus improving contamination safety for the health care team involved in handling and irradiating the blood.

The advantage of these further embodiments, as with the first embodiment described above, is that the blood remains fixed or stationary within the vessel in which it is irradiated. The light source is LED, which provides instant cooler light and the ability to select wavelengths. A computer program is provided that controls the operation of the unit, allowing double-blind studies, and data can be transmitted back to a computer for processing. The program controls intensity, timing, duration, wavelengths of light emission and other data to be stored or transmitted to a processor for further processing. Energy usage from the LEDs is low enough to allow portable units for disaster relief and field hospitals. Safety is enhanced because light cannot escape from the unit and damage eyes.

In operation, the space between the hollow core and the quartz tube is filled with blood, then disconnected from the patient and the tube is placed inside the irradiation chamber (62, 92). This offers greater safety to the patient by eliminating the chance of electrocution. Alternatively, valves and hoses may be used as in the first embodiment to couple to the patient and to the tube 74, as described above in the first embodiment.

Ideally, the quartz tube has a 30 millimeter interior diameter and contains a 20 millimeter cylindrical core of solid plastic that is reflective, the purpose of which is merely to take up space inside the quartz tube and to provide another reflective surface. The size of the inner reflective core may be altered to produce tubular cassettes of varying volumes.

From the foregoing it will be appreciated that, although specific embodiments of the invention have been described herein for purposes of illustration, various modifications may be made without deviating from the spirit and scope of the invention. Accordingly, the invention is not limited except as by the appended claims and the equivalents thereof.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A device for subjecting a patient's blood to light for the purpose of altering the immune function of the patient, the device comprising:
a chamber having a window formed of a quartz plate and adapted to receive blood intravenously from the patient;
a back attached to the chamber and formed of hard plastic, the back having an inlet port that communicates with the chamber, including a stopcock valve adjacent the outlet port to retain blood in the chamber stationary during treatment of the blood and to selectively permit the entry of fluids into the chamber when treated blood exits the chamber back to the patient via the inlet port;
a housing for directing light from a light source to the chamber; and
a controller that activates the light source to treat the stationary blood in the chamber and to deactivate the light source to stop the treatment.

2. The device of claim 1, wherein the housing comprises a holder made of plastic having a slot to receive the chamber, a clamp to hold the chamber in the housing, a mounting with a light source board at an end opposite the holder, and a reflective inner surface adapted to be in contact with the patient's blood to reflect light from the light source to the chamber.

3. The device of claim 1 wherein the light source comprises a printed circuit board having an array of light emitting diodes, at least one of which is an ultraviolet light emitting diode.

4. A method of treating blood from a patient to alter the immune function of the blood, comprising:
a. receiving blood intravenously from a patient at an inlet port of a chamber by force of the intravenous blood pressure;
b. filling the chamber with the patient's blood from the inlet port at the bottom of the chamber to a valve at an outlet port at the top of the chamber;
c. exposing the blood in the chamber to a light source by activating the light source as the blood is held stationary in the chamber for the purpose of altering the immune function of the blood of the patient;
d. deactivating the light source and opening the valve at the outlet port to introduce fluids into the chamber through the outlet port with sufficient force to return the blood back to the patient intravenously and flushing the chamber with the fluid; and
repeating steps a through d.

5. A device for irradiating blood to alter its immune function, comprising:
a chamber having an elongate configuration defining a hollow interior, the chamber having an opening to provide access to the chamber and a cover to close the opening;
a tubular cassette sized and shaped to have an elongate configuration and to be received in the interior of the chamber, the cassette having an inside diameter sized and shaped to receive a reflective core having an elongate configuration of an exterior diameter that is smaller than the interior diameter of the cassette to provide a space for receiving and holding blood stationary; and
an array of light-emitting members arranged on the chamber for emitting light of at least one wavelength into the interior of the chamber for irradiating and treating the blood held stationary inside the cassette to alter the immune function of the blood; and
a controller coupled to the light source to activate the light source and treat the blood when the blood is stationary in the interior of the cassette and to deactivate the light source to stop treating the blood in the interior of the cassette.

6. A device for irradiating a predetermined stationary quantity of a patient's blood to alter the immune function of the blood, comprising:
an outer wall enclosing an interior;
a container configured to hold the predetermined quantity of blood stationary in the interior of the outer wall;
a light source for emitting light onto the blood to irradiate the blood to alter the immune function of the blood; and
a controller coupled to the light source to activate the light source when the blood is stationary in the interior of the cassette and to deactivate the light source to stop treating the blood in the interior of the cassette.

7. The device of claim 6, wherein the outer wall is configured to enable the container to be removably placed in the interior.

8. The device of claim 7, comprising a door formed in the outer wall sized and shaped to allow the container to be placed into and removed from the interior.

9. The device of claim 7, wherein the container is formed of quartz material to admit light to the blood in the container.

10. The device of claim 7, wherein the container comprises a core having an exterior surface and at least a portion of the exterior surface having reflective characteristics.

11. The device of claim 10, wherein the core is configured to be removable from the container.

12. The device of claim 10, wherein the exterior surface of the core and an interior surface of the container wall form a space to hold the blood.

13. The device of claim 10, wherein at least a portion of the container comprises a wall formed of material that admits light from the light source.

14. The device of claim 6, wherein the outer wall is formed of stainless steel, and the light source is attached to the exterior of the outer wall, the outer wall comprising an opening to admit light from the light source to the interior.

15. A device for treating fluids, comprising
a chamber formed by an outer wall having an inside reflective surface;
a reflective core positioned inside the outer wall for direct contact with the fluid;
an intermediate wall positioned between the outer wall and the reflective core and configured to form a fluid container to hold a predetermined quantity of fluid stationary;
at least one light-emitting device configured to emit light through the intermediate wall for irradiating fluid in the fluid container; and
a controller that activates the at least one light emitting device to treat the stationary fluid in the fluid container and to deactivate at least one light emitting device to stop treating the blood.

16. The device of claim 15, wherein the intermediate wall is formed of quartz material and configured to admit light therethrough.

17. The device of claim 16, wherein the intermediate wall and the core form a removable container configured to be detached from the chamber.

18. The device of claim 16, wherein the light emitting device comprises at least one light-emitting diode configured to emit ultraviolet light.

19. A method for treating fluid, comprising:
placing fluid inside a container formed by a reflective core and a circumscribing quartz wall defining a space between the circumscribing wall and the core for retaining the fluid in a stationary condition and in direct contact with the reflective core;
placing the container inside a chamber formed by an outer wall having reflective characteristics;
exposing the container and the fluid to light to treat the fluid as the fluid is held stationary inside the container; and
controlling the light to activate the light to treat the fluid when the fluid is held stationary and to deactivate the light to stop treating the fluid.
